(19) United States
(12) Patent Application Publication
(10) Pub. No.: US 2022/0249293 A1
(43) Pub. Date: Aug. 11, 2022

(54) WOUND TREATMENT SYSTEM

(71) Applicants: BenQ Materials Corporation, Taoyuan City (TW); National Cheng Kung University, Tainan City (TW)

(72) Inventors: Chih-Lung LIN, Tainan City (TW); Feng-Ching CHENG, Tainan City (TW); Shih-Wei CHAO, Tainan City (TW)

(21) Appl. No.: 17/457,432

(22) Filed: Dec. 2, 2021

(30) Foreign Application Priority Data
Feb. 9, 2021 (TW) 110105152

(51) Int. Cl. A61F 13/02; A61N 5/06; A61M 1/00

(52) U.S. Cl. CPC A61F 13/0216; A61N 2005/0662; A61M 1/91; A61N 5/0613

(57) ABSTRACT

A wound treatment system includes a wound dressing, a negative-pressure source, a light-emitting device, an image recognition module, and a control module. The wound dressing includes a housing with an opening, and a barrier layer with a conduit pathway separating the inside of the housing from the opening to form a receiving space. The negative-pressure source is fluidly connected to the wound dressing to remove tissue exudate of the wound. The light-emitting device is disposed in the receiving space, optically coupled to the barrier layer and can emit light of different wavelength bands as the detection light required by the image recognition module disposed on a side of the opening of the housing of the wound dressing and provide specific wavelengths of phototherapy required by different stages of the wound.

### CROSS-REFERENCE TO RELATED APPLICATION

This application claims priority to Taiwan Application Serial Number 110105152, filed Feb. 9, 2021, which is herein incorporated by reference.

### BACKGROUND

### Field of Invention

The present invention relates to a wound treatment system and particularly, relates to a chronic wound treatment system for treating different wound healing phase of chronic wound sites with different wavelength-band light and negative treatment environment.

### Description of Related Art

Wound healing is a complicated and continuous process and begins immediately following skin injury. The wound healing can be divided into four phases, hemostasis; inflammation, proliferation and wound remodeling. In hemostasis stage, the blood flows to the wound site to deliver the protein and other hemostasis materials to make a clot to prevent further blood loss. In inflammation phase, the immune system resists the bacteria and pathogens infection, the white blood cells engulf damaged cells along with bacteria and other pathogens or debris from the wound area. This inflammation process causes the wound site slightly red, swollen, hot, and painful. In the proliferation phase, the new vascular tissue and granulation tissue grows and rebuilds. Lastly, the epidermal tissue of the wound site is remodeled and closed.

However, the chronic wounds, such as diabetic ulcers, pressure ulcers or venous or arterial ulcers caused by poor peripheral blood circulation, has failed to follow the wound healing stages as defined and the wound infection is reoccurred between the inflammation stage and the proliferation stage. Thus, the wound healing delays into the remodeling stage. For treating such a chronic wound, it is suggested to remove the excess exudate from the wound site via application of negative pressure. FIG. 1 shows a schematic diagram of a known wound dressing used with a negative pressure wound treatment system used in the related art. The known wound dressing 1 for a negative pressure wound treatment system comprises a wound contact layer 12 for covering the wound site 15; an absorption layer 13 for absorbing the exudate from the wound site 15; and a water barrier layer 14 to seal the outer side of the absorption layer 13 to prevent the exudate from contacting with the environment. A negative pressure is applied to the wound dressing 1 via a tube 11 fluidly communicated with a negative pressure source (not shown in the drawings) to suction excess exudate from the wound site but keep the wet environment of the wound site without infiltration. A chronic wound site cannot keep an aseptic condition under a negative pressure environment and covered by a wound dressing. Thus, the wound healing process may be relied on the individuals' immune resistance or application of antibiotics. Furthermore, for maintaining the wet environment of the wound site, the exudate may help the growth of the bacteria or pathogens to influence the wound healing process. In such case, a further assistant system is required to inhibit the growth of the bacteria or pathogens and accelerate the growth of the cell proliferation.

The inventors of the present invention provide a wound treatment system integrating a negative pressure treatment and phototherapy for chronic wounds. The present wound treatment system can apply a negative pressure and a wavelength-band light controlled and conditioned automatically in accordance with the wound healing phase of the wound area for accreting the wound healing.

### SUMMARY

In accordance with the above mentioned, the present invention is to provide a wound treatment system with patentability including novelty, non-obviousness, industrial applicability and the like to overcome the difficulty of the present products.

The present invention is to provide a wound treatment system. The wound treatment system comprises a wound dressing comprising a housing with an opening, a wound contact layer disposed at the opening to cover a wound area and a barrier layer disposed between a housing interior area and the wound contact layer at the opening to separate a receptacle space; wherein the barrier layer comprises at least one conduit pathway with openings at a receptacle space side and a wound contact layer side; a negative pressure source comprising a negative pressure transmission tube to fluidly communicate with the receptacle space of the housing of the wound dressing for applying a negative pressure via the negative pressure transmission tube to the wound area for suctioning an exudate from the wound area through the wound contact layer via the conduit pathway to the receptacle space; a light source disposed in the receptacle space of the wound dressing and light coupling to the barrier layer for emitting a first wavelength-band light and a second wavelength-band light; an image recognition module disposed at a side wall of the opening of the wound dressing for receiving an image light reflected by a light emitting from the light source and irradiating the wound area; and a control module electrically connected to the negative pressure source, the light source and the image recognition module.

In an embodiment of the present wound treatment system, the receptacle space of the wound dressing comprises an absorption layer, and the negative pressure transmission tube further comprises an air-permeable water barrier layer to avoid the exudate flowing into the negative pressure source.

In an embodiment of the present wound treatment system, a storage means is further arranged between the wound dressing and the negative pressure source and fluidly communicates with the receptacle space of the housing of the wound dressing and the negative pressure source via the negative pressure transmission tube.

In another embodiment of the present wound treatment system, a wavelength of the first wavelength-band light can be from 350 nm to 500 nm, a wavelength of the second wavelength-band light can be from 600 nm to 850 nm.

In further another embodiment of the present wound treatment system, the light source can emit a third wavelength-band light with a wavelength from 500 nm to 600 nm.

In still another embodiment of the present wound treatment system, the light source can be sequentially emitted the first wavelength-band light, the second wavelength-band light and the third wavelength-band light or a blend thereof.

In still another embodiment of the present wound treatment system, the light source can be a point light array.

In still another embodiment of the present wound treatment system, the light source is driven by a constant voltage or a constant current.

In still another embodiment of the present wound treatment system, the image recognition module comprises a receiving unit for receiving a real-time wound image via the light emitted from the light source and an output unit for transmitting the real-time wound image to the control module.

In still another embodiment of the present wound treatment system, the control module comprises a driving means to drive the negative pressure source, the light source and the image recognition module; a storage means for storing a database containing a plurality of classified wound-site images; and a processing means for evaluating and determining a wound-healing phase of a wound site and outputting the information to the driving means after receiving the real-time would image of the wound site from the image recognition module and comparing the real-time wound image with the plurality of classified wound-site images in the database of the storage means.

It is to be understood that both the foregoing general description and the following detailed description are by examples, and are intended to provide further explanation of the invention as claimed.

### BRIEF DESCRIPTION OF THE DRAWINGS

The accompanying drawings are included to provide a further understanding of the invention, and are incorporated in and constitute a part of this specification. The drawings illustrate embodiments of the invention and, together with the description, serve to explain the principles of the invention. In the drawings,

FIG. 1 is a schematic view of a wound dressing used with a known negative pressure treatment system.

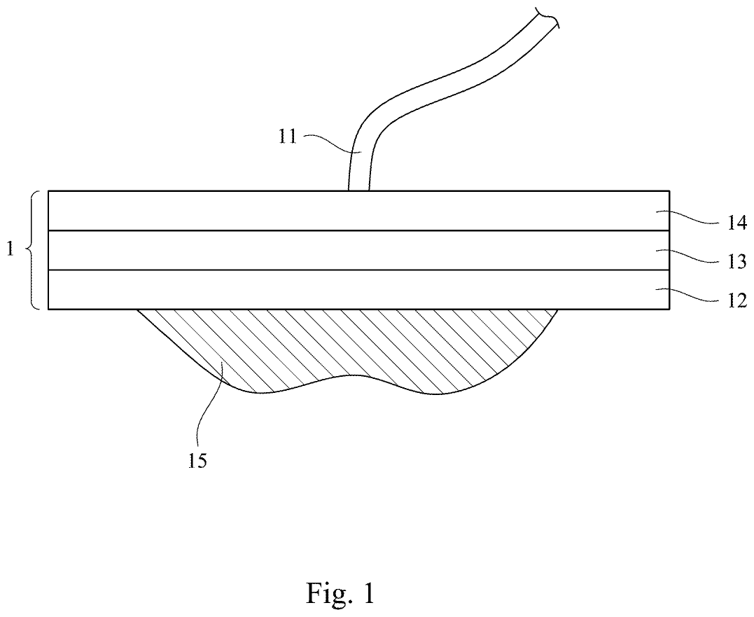

FIG. 2 is a schematic view of an embodiment of the wound treatment system of the present invention.

FIG. 3A is a schematic view of an embodiment of the wound dressing of the wound treatment system of the present invention.

FIG. 3B is a schematic view of another embodiment of the wound dressing of the wound treatment system of the present invention.

FIG. 4 is a schematic view of further another embodiment of the wound treatment system of the present invention.

FIG. 5 is a schematic view of an embodiment of the light source used in a wound treatment system of the present invention.

FIG. 6 is a diagram showing another embodiment of the wound treatment system of the present invention.

FIG. 7 is a schematic flow chart diagram illustrating the wound treatment methods of the present invention.

### DETAILED DESCRIPTION

With reference to the following more detailed description and claims taken in conjunction with the accompanying drawings. The purpose of the drawings is only for illustrating the present invention and may not exhibit the true proportions and precise configuration. Thus, the drawings cannot be used limit the concept and scope of the present invention. The present disclosure is only defined by the appended claims.

The following description together with the accompanying drawings is to illustrate embodiments of the wound treatment system of the present invention. For understanding, the same elements in the following embodiments are described with the same symbols.

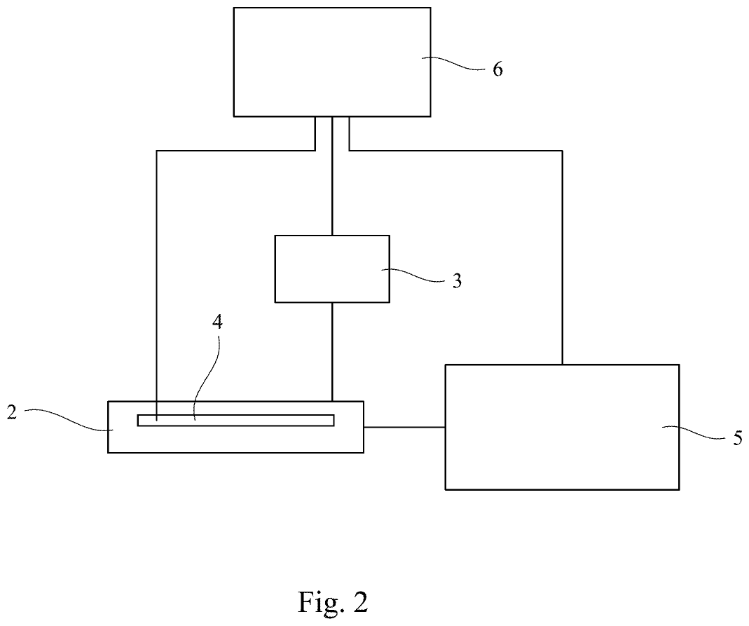

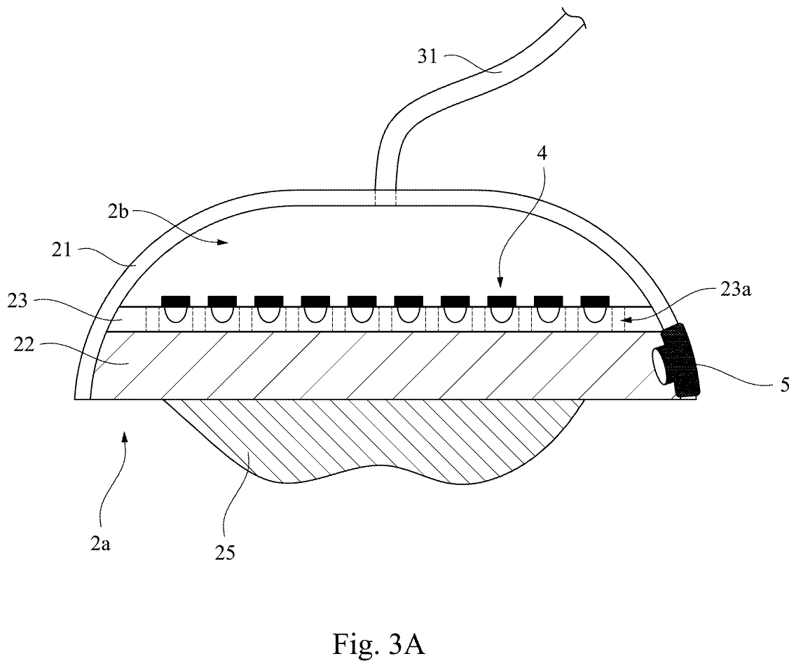

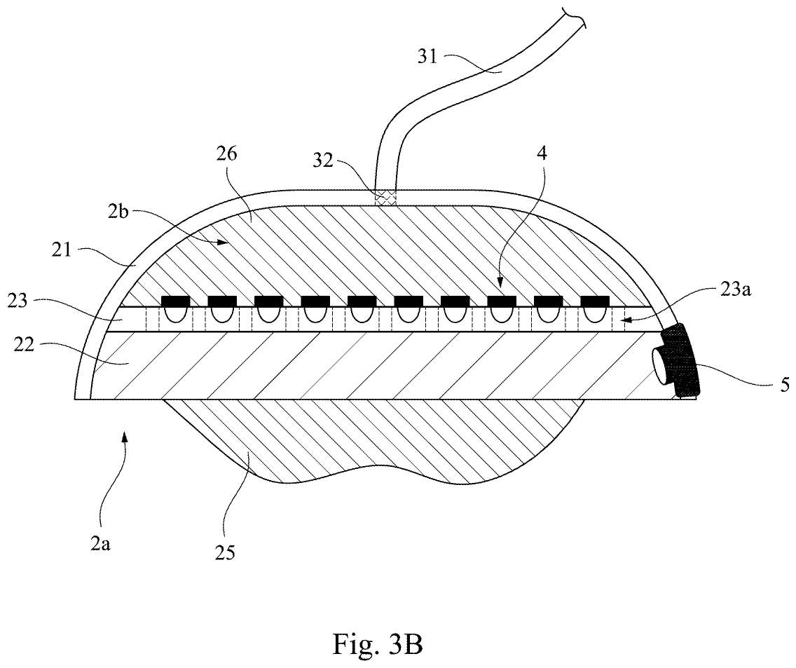

Referring to FIGS. 2 and 3A illustrating an embodiment of the wound treatment system of the present invention, the wound treatment system comprises a wound dressing 2 comprising a housing 21 with an opening 2a, a wound contact layer 22 disposed in the opening 2a to cover a wound area 25, and a barrier layer 23 to separate the interior space of the housing 21 and from the wound contact layer 22 at the opening 2a to form a receptacle space 2b, wherein, the barrier layer 23 comprises a conduit pathway 23a with an opening at the receptacle space 2b and another opening at the wound contact layer 22; a negative pressure source 3 comprising a negative pressure transmission tube 31 to fluidly communicate with the receptacle space 2b of the housing 21 of the wound dressing 2 for applying a negative pressure via the negative pressure transmission tube 31 to the wound area 25 for suctioning the exudate from the wound area 25 through the wound contact layer 22 via the conduit pathway 23a to the receptacle space 2b; a light source 4 disposed at the receptacle space 2b of the wound dressing and coupled to the barrier layer 23 to emit a first wavelength-band light and a second wavelength-band light; an image recognition module 5 disposed at the side wall of the opening 2a of the housing 21 of the wound dressing 2 for receiving the image light reflected by the light emitting from the light source 4 and irradiating the wound area 25; and a control module 6 electrically connecting to the negative pressure source 3, the light source 4 and the image recognition module 5.

Referring to FIG. 3B, in an embodiment of the wound treatment system of the present invention, the receptacle space 2b of the wound dressing 2 comprising an absorption layer 26 to absorb and store the exudate. The absorption layer 26 can be made by, for example, such as polymer fiber, fiber sponge, foam material, super absorbent polymer, hydrogel material or gel material to be contained in the receptacle space 2b. The negative pressure transmission tube 31 further comprises an air-permeable water barrier 32, which is a moisture permeable film or a polymer microporous film made by for example, polyethylene, polypropylene, polyvinyl alcohol, polyvinyl acetate, polytetrafluoroethylene, polyvinylidene fluoride, polyvinylpyrrolidone, polyurethane, polyamide, polyester, polyacrylate , polymethacrylate or polypropylene amide to prevent the exudate suctioned by negative pressure from flowing into the negative pressure source 3 to cause damage and contamination thereto.

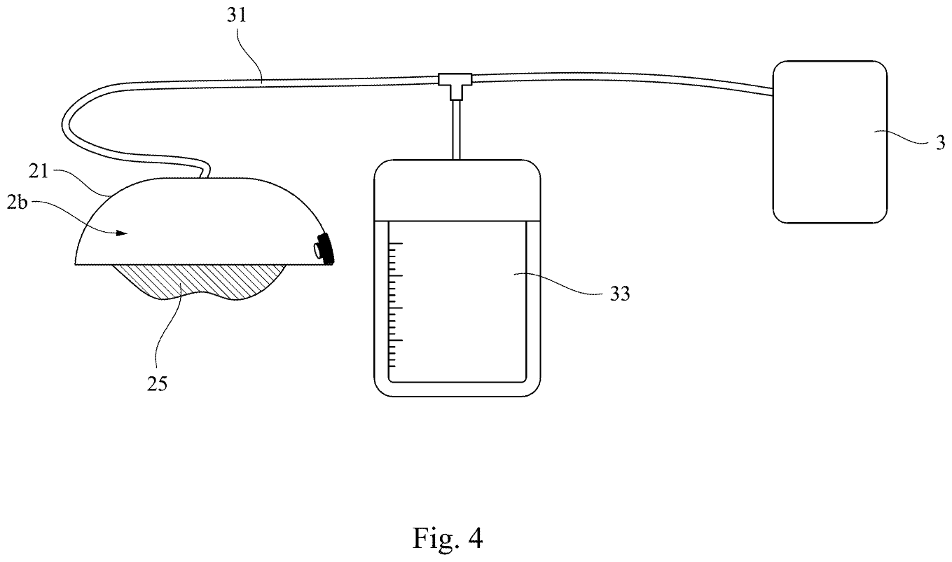

Referring to FIG. 4, in an embodiment of the present wound treatment system, a storage means 33 is further arranged between the wound dressing 2 and the negative pressure source 3 and fluidly communicates with the receptacle space 2b of the housing 21 of the wound dressing 2 and the negative pressure source 3 via the negative pressure transmission tube 31. The use of the storage means 33 is dependent on the condition of the wound areas. The storage means 33 is for storing large and excessive amount of exudate from the wound area for preventing the large amount of exudate from remaining close to the wound area 25 to increase the potential infection due to the growth of bacteria and from the negative pressure unevenly distributed and transmission caused by the blockage of the excessive exudate.

The barrier layer 23 can be made by water barrier materials, such as, polyolefin, polyvinyl acetate, polyvinylpyrrolidone, polyurethane, polyimide, polyester, polyacrylate, polymethacrylate, or polycarbonate and the likes. The materials for the barrier layer 23 is preferred no color and transparent for not affecting the light transmittance and color shifting when coupling to light source 4. The wound contact layer 22 is made by the material with porous structure, such as flexible polymer fibers, silicone, hydrogel and the like for not irritating the wound area 25 and enables the exudate to get through the conduit pathway 23a of the barrier layer 23 into the receptacle space 2b. The refraction index of the materials of the wound contact layer 22 is adjusted to meet with the refraction index of the materials of the barrier layer 23 in order for the light with different wavelength emitted from the light source 4 optically coupling to barrier layer 23 to be advantageously transmitted to the wound area 25 to provide phototherapy to adjuvantly heal the wound. The image recognition module 5 is disposed at the side wall of the opening 2a of the housing 21 of the wound dressing 2 for receiving the image light reflected by the light emitting from the light source 4 and irradiating the wound area 25 to formulate a real-time wound image for determining the condition of the wound area 25.

In an embodiment of the present wound treatment system, the wavelength of the first wavelength-band light of the light source 4 can be from 350 nm to 500 nm close to blue light band, the wavelength of the second wavelength-band light can be from 600 nm to 850 nm close to red light to near-infrared light band. It is known that red light to near-infrared light band can stimulate and increase the fibroblasts proliferation which plays an important role in wound healing. The blue light band is known to lower the average survival rate of Pseudomonas aeruginosa and other bacteria which commonly induce the inflammation of wound area.

In another embodiment of the present wound treatment system, the light source 4 can further emit a third wavelength-band light with a wavelength from 500 nm to 600 nm close to green light band. Although the green light is not adjuvant to wound healing, the light in green light band is together with red wavelength band and blue wavelength band to complete the wavelength band to a visible light. Thus, the image recognition module 5 can catch a better real-time wound area image for the control module to evaluation. The wound area 25 can be precisely evaluation by the different color of the wound area 25, for example, the red granulation tissue with sufficient oxygen, inflammatory yellow or white dead tissue and necrotic eschar-black tissue and by the area percentage of each color. In addition, the light source 4 can emit the first wavelength-band light, the second wavelength-band light and the third wavelength-band light in sequence to obtain the real-time image of the wound area under single wavelength-band light without intervention and reconstruct the images to show as a visible-spectrum image. The light source can emit the light in blend to directly obtain a full-spectrum real-time image of the wound area.

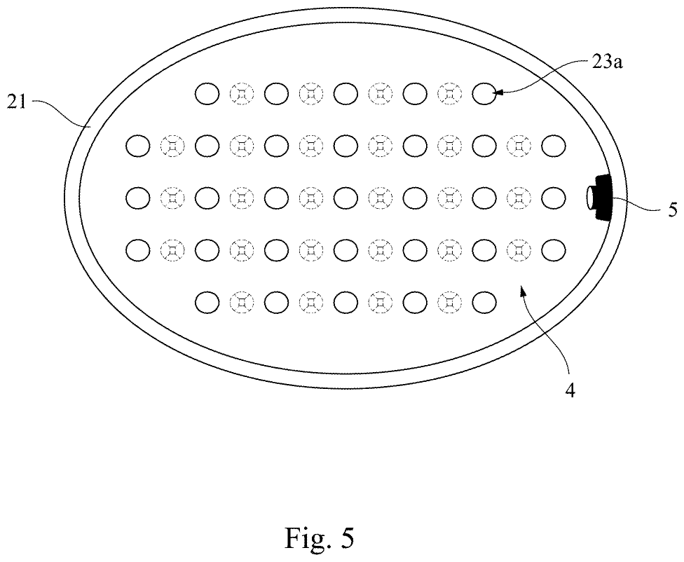

Referring FIG. 5 illustrating another embodiment of the present wound treatment system, the light source 4 is a point light array. The point light array can be a plurality of single-light laser, light-emitting diode or organic light-emitting diode with gas-moisture barrier packages. Thus, the point lights of the point light array can be alternately arranged with the conduit pathway of the barrier layer 23 without affecting the negative pressure therapy treatment. Since the excitation light source is not a whole visible light band and simple to obtain a single-color light source of narrower full width at half maximum (FWHM), the point light array can be designed a suitable arrangement of the point light array in accordance with the area and the depth of the wound area, the wound area image received by the image recognition module 5 to be reconstructed to be a high resolution real-time image of wound area.

In another embodiment of the present wound treatment system, the light source 4 can use the local dimming technology commonly in the backlight module of the liquid crystal display to emit a stable illumination, driven by constant voltage or constant current drivers, to conduct phototherapy or catch the real-time image of the wound area. In the phototherapy stage, the illumination of the light source 4 is preferred from 5 mW/cm² to 25 mW/cm² and the radiant energy density is from 1 J/cm² to 5 J/cm² for providing sufficient therapy benefits and avoiding irritating the wound area without skin protection.

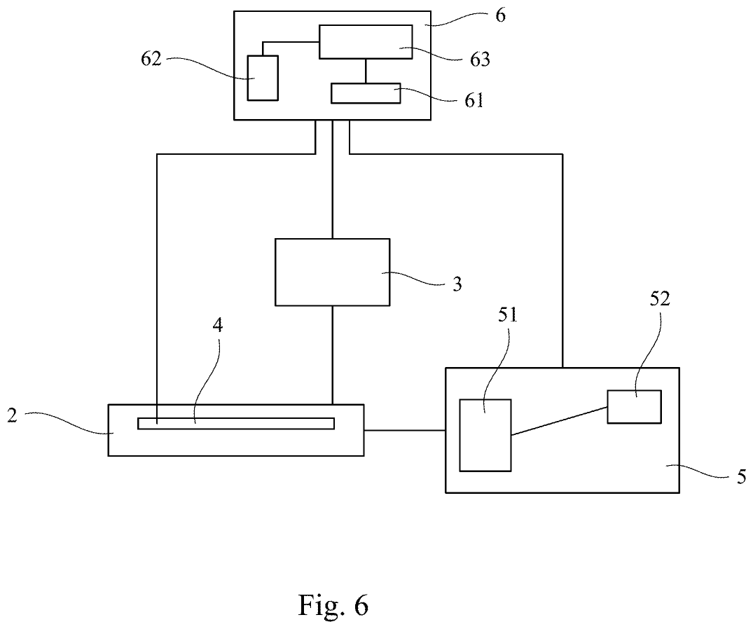

Referring FIG. 6 which illustrating another embodiment of the present wound treatment system, the image recognition module 5 comprises a receiving unit 51 for receiving real-time wound image via the light emitted from the light source 4 and an output unit 52 for transmitting the real-time wound image to the control module 6.

In still another embodiment of the present wound treatment system, the control module 6 comprises a driving means 61 to drive the negative pressure source 3, the light source 4 and the image recognition module 5; a storage means 62 for storing a database containing a plurality of classified wound-site images; and a processing means 63 for evaluating and determining the wound-healing phase of the wound site and outputting the signal to the driving means 61 after receiving the real-time image of the wound site from the image recognition module 5 and comparing the received image with the images classified and stored in the database of the storage means 62.

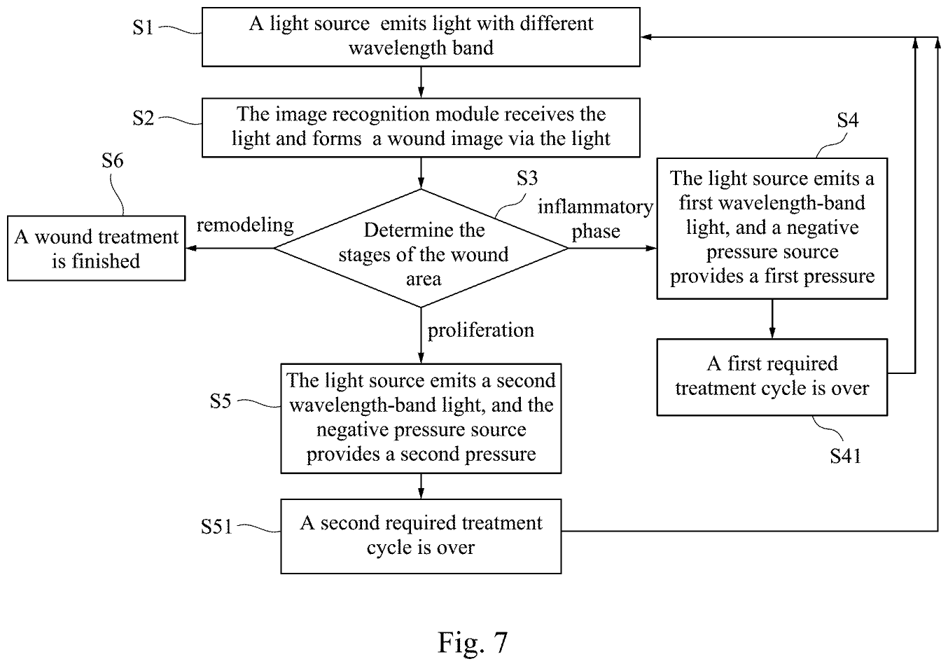

Referring to FIGS. 6 and 7. FIG. 7 is a schematic flow chart diagram illustrating the wound treatment methods of the present invention.

In Step S1, the driving means 61 of the control module 6 drives the light source 4 to emit light with different wavelength band, for example, the first wavelength-band light and the second wavelength-band light, to provide light source for the image recognition module.

In Step S2, the driving means 61 of the control module 6 drives the image recognition module 5, the receiving unit 51 receives the light emitted from the light source 4 and an output unit 52, forms a real-time wound image and further transmits the real-time wound image to the control module 6.

In Step S3, the processing means 63 of the control module 6 compares the received image with the images classified and stored in the database, evaluates and determines that the features of the wound area are classified into inflammation phase, proliferation or remodeling in wound-healing phase, outputs the information to the driving means 61.

In Step S4, when the processing means 63 evaluates and determines that the features of the received real-time image of the wound area meet the image features of the inflammatory phase of a wound site in the database, the driving means 61 drives the light source 4 to emit the first wavelength-band light, blue light, to inhibit the bacterial growth and drives the negative pressure source 3 to provide a first pressure to the wound dressing 2.

In Step S41, the light emitted and the negative pressure set at Step S4 are maintained at a stable value for a period of time until the first required treatment cycle for the inflammatory phase is over. Then, Step 51 is repeated to evaluate the condition of the wound area and determine if another treatment cycle is needed.

In Step S5, when the processing means 63 determines that the features of the received real-time image of wound area meet the image feature of a wound area in proliferation phase in the database of the storage means 62, the driving means 61 drives the light source 4 to emit the second wavelength-band light, red and near-infrared light, to enhance the fibroblast proliferation and to drive the negative pressure source 3 to provide a second pressure to the wound dressing 2. Because the amount of the exudate occurred in proliferation phase is less than that occurred in the inflammatory phase, the second pressure can be less than the first pressure required for the inflammatory phase to ease patients' discomfort.

In Step S51, the light emitted and the negative pressure set at Step S5 are maintained at a stable value for a period of time until the second required treatment cycle for the proliferation phase is over. Then, Step S1 is repeated to evaluate the condition of the wound area and determine if another treatment cycle is needed.

In Step S6, when the processing means 63 determines that the features of the received real-time image of wound area meet the image feature of a healed wound area in the database of the storage means 62, the wound treatment is finished.

Although particular embodiments have been shown and described, it should be understood that the above discussion is not intended to limit the present invention to these embodiments. Persons skilled in the art will understand that various changes and modifications may be made without departing from the scope of the present invention as literally and equivalently covered by the following claims.

What is claimed is:

1. A wound treatment system comprising
a wound dressing comprising
a housing with an opening,
a wound contact layer disposed at the opening to cover a wound area and
a barrier layer disposed between a housing interior area and the wound contact layer at the opening to separate a receptacle space;
wherein the barrier layer comprises at least one conduit pathway with openings at a receptacle space side and a wound contact layer side;
a negative pressure source comprising a negative pressure transmission tube to fluidly communicate with the receptacle space of the housing of the wound dressing for applying a negative pressure via the negative pressure transmission tube to the wound area for suctioning an exudate from the wound area through the wound contact layer via the at least one conduit pathway to the receptacle space;
a light source disposed in the receptacle space of the wound dressing and light coupling to the barrier layer for emitting a first wavelength-band light and a second wavelength-band light;
an image recognition module disposed at a side wall of the opening of the wound dressing for receiving an image light reflected by a light emitting from the light source and irradiating the wound area; and
a control module electrically connected to the negative pressure source, the light source and the image recognition module.

2. The wound treatment system as claimed in claim 1, wherein the receptacle space of the wound dressing comprises an absorption layer, and the negative pressure transmission tube further comprises an air-permeable water barrier layer to avoid the exudate flowing into the negative pressure source.

3. The wound treatment system as claimed in claim 1, wherein a storage means is further arranged between the wound dressing and the negative pressure source and fluidly communicates with the receptacle space of the housing of the wound dressing and the negative pressure source via the negative pressure transmission tube.

4. The wound treatment system as claimed in claim 1, wherein a wavelength of the first wavelength-band light is from 350 nm to 500 nm, a wavelength of the second wavelength-band light is from 600 nm to 850 nm.

5. The wound treatment system as claimed in claim 4, wherein the light source further emits a third wavelength-band light with a wavelength from 500 nm to 600 nm.

6. The wound treatment system as claimed in claim 5, wherein the light source is sequentially emitted the first wavelength-band light, the second wavelength-band light and the third wavelength-band light.

7. The wound treatment system as claimed in claim 1, wherein the light source is a point light array.

8. The wound treatment system as claimed in claim 1, wherein the light source is driven by a constant voltage or a constant current.

9. The wound treatment system as claimed in claim 1, wherein the image recognition module comprises a receiving unit for receiving a real-time wound image via the light emitted from the light source and an output unit for transmitting the real-time wound image to the control module.

10. The wound treatment system as claimed in claim 9, wherein the control module comprises
a driving means to drive the negative pressure source, the light source and the image recognition module;
a storage means for storing a database containing a plurality of classified wound-site images; and
a processing means for evaluating and determining a wound-healing phase of a wound site and outputting an information to the driving means after receiving the real-time wound image of the wound site from the image recognition module and comparing the real-time wound image with the plurality of classified wound-site images in the database of the storage means.
